(19) United States
(12) Patent Application Publication
(10) Pub. No.: US 2025/0147031 A1
(43) Pub. Date: May 8, 2025

(54) SYSTEMS AND METHODS FOR BIOLOGICAL OPTICAL IMAGING WITH ARTIFICIAL PARTICLES REFERENCES

(71) Applicant: IDEXX Laboratories, Inc., Westbrook, ME (US)

(72) Inventors: Jeremy Hammond, Standish, ME (US); Jason Aguiar, Freeport, ME (US); Lucy Ericson, Gorham, ME (US); Tim Butcher, Windham, ME (US); Jui-Ming Lin, Falmouth, ME (US)

(73) Assignee: IDEXX Laboratories, Inc., Westbrook, ME (US)

(21) Appl. No.: 18/940,011

(22) Filed: Nov. 7, 2024

Related U.S. Application Data
(60) Provisional application No. 63/597,095, filed on Nov. 8, 2023.

(51) Int. Cl. G01N 33/58; G01N 33/543; G01N 33/551

(52) U.S. Cl. CPC G01N 33/582; G01N 33/54386; G01N 33/551

(57) ABSTRACT

Systems and methods for use with a biological sample include a microscopy device, a diluent, a sample holder, and one or more of lyophilized cakes comprising artificial particles. The lyophilized cakes and the biological sample are mixed with the diluent in the sample holder to form a solution. The biological sample is imaged with the artificial particles as reference markers using the microscopy device. A settling time of the artificial particles is shorter than or equal to a settling time of the biological sample.

### CROSS REFERENCE TO RELATED APPLICATION

This application claims priority to U.S. Provisional Patent Application Ser. No. 63/597,095, filed on Nov. 8, 2023, entitled “SYSTEMS AND METHODS FOR BIOLOGICAL OPTICAL IMAGING WITH ARTIFICIAL PARTICLES REFERENCES,” the entire contents of which are incorporated by reference in the present disclosure.

### TECHNICAL FIELD

The present specification relates to optical microscopy imaging, and more particularly, to optical microscopy imaging using reference particles.

### BACKGROUND

Dynamic imaging of biological samples using optical microscopy devices involves biological sample preparation and real-time tracking of the biological samples, such as cells or tissues. The viability of the biological samples and post-treatment on the biological samples demands a fast and accurate imaging process and technology. Accordingly, there is a desire to use artificial particles as a reference to enhance the dynamic imaging capacity of the optical microscopy device.

### SUMMARY

In embodiments, a method for use with a biological sample includes mixing artificial particles and the biological sample with a diluent to form a solution, conducting focus sequences with the artificial particles as reference markers in the solution using a microscopy device, and imaging the biological sample in the solution using the microscopy device. The artificial particles are suspended in the solution independent from the biological sample. A settling time of artificial particles is shorter than or equal to a settling time of the biological sample.

In another embodiment, a system for use with a biological sample includes a microscopy device, a diluent, a sample holder, and one or more of lyophilized cakes comprising artificial particles. The lyophilized cakes and the biological sample are mixed with the diluent in the sample holder to form a solution. The biological sample is imaged with the artificial particles as reference markers using the microscopy device. A settling time of the artificial particles is shorter than or equal to a settling time of the biological sample.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 schematically depicts an example biological optical imaging system, according to one or more embodiments shown and described herein;

FIG. 2 schematically depicts example components of a controller of the microscopy device, according to one or more embodiments shown and described herein;

FIG. 3 schematically depicts a solution in a sample holder, according to one or more embodiments shown and described herein;

FIG. 4 schematically depicts example imaging modes using the microscopy device, according to one or more embodiments shown and described herein

FIG. 5 schematically depicts example artificial particles suspended in the solution, according to one or more embodiments shown and described herein;

FIG. 6 visually depicts example distributions of artificial particles suspended in the solution, with and without any lyophilization treatments, according to one or more embodiments shown and described herein;

FIG. 7 depicts a flowchart of an example method of biological sample imaging using the biological optical imaging system, according to one or more embodiments shown and described herein;

FIG. 8 depicts a flowchart of an example method of the determination of biological sample settlement using the biological optical imaging system, according to one or more embodiments shown and described herein;

FIG. 9 depicts a flowchart of an example method of confirmation of reagent using the biological optical imaging system, according to one or more embodiments shown and described herein; and

FIG. 10 depicts a flowchart of an example method of confirmation of acellular using the biological optical imaging system, according to one or more embodiments shown and described herein.

### DETAILED DESCRIPTION

Imaging technology holds a prominent role in modern biological laboratories, facilitating detailed examinations of biological phenomena across various scales and levels of complexity. The emergence of digital microscopy has ushered in a new era of optical imaging, providing researchers with the means to closely monitor and analyze biological samples with unprecedented precision in terms of resolution, specificity, dimensionality, and scale. Within the realm of digital microscopy, techniques such as fluorescent microscopy and bright field microscopy, specifically tailored for biological and medical sample analysis, have become invaluable tools. Nevertheless, a foundational concern when employing these advanced imaging techniques is the necessity to maintain the viability and biological relevance of the specimens under investigation.

In practice, microscopy systems often accommodate biological samples within a reagent solution, which may include liquid diluents supplemented with fluorescent stains. These systems are intricately designed to capture focused images of biological samples and cells while effectively excluding undesired or irrelevant elements, such as, without limitations, lipids, pollen, grass, dirt, dust, and talc (e.g. from gloves), to ensure the accuracy of the analysis. Intriguingly, the viability of the samples can occasionally depend on the behavior of the fluorescent stains post-incubation, as the kinetics of the staining reaction may introduce changes in fluorescence that provide valuable insights. For example, these fluorescence changes can play a pivotal role in cell classification, characterization, and the assessment of cellular responses to various stimuli. Thus, the integration of digital microscopy and advanced staining techniques underscores the dynamic and multifaceted nature of contemporary biological research, where technological advancements continue to shape the way we explore and understand life's intricacies.

To enhance the real-time identification, imaging, and characterization of the microscopy, it is desirable to enhance the feasibility of fast and accurate imaging while maintaining the viability of the biological samples. The systems and methods disclosed herein utilize a size reference of artificial particles, such as quality control (QC) beads. The artificial particle references provide qualitative and quantitative understandings of the size distribution of the cells of interest. For example, the system may automatically provide the size distribution of lymphocytes to discern small, intermediate, and large cells that can then be used to evaluate lymphomas or reactive lymph nodes.

The artificial particles discussed herein can be incorporated into cakes, which may consist of particles such as polystyrene and polyacrylate microspheres. The cakes may be lyophilized cakes. The reagent may contain nucleic acid staining dyes for visualizing cell morphology, antibodies for labeling cell surface markers, or proteins for analyzing enzymatic reactions. These chemical and biological materials necessitate specific storage conditions, particularly at temperatures below 4° C., to preserve their stability. Storing the reagent's particles in a cold environment, either in liquid or solid form, can lead to alterations in particle morphology or aggregation, thereby affecting reference performance. Conversely, particles encapsulated with fluorescence or magnetite may also pose stability risks when stored at room temperature. To mitigate these risks and maintain the integrity of various particle types, the method involves lyophilizing the particles in a liquid suspension to create a stable dry matrix. Additionally, the liquid suspension to form the cakes are lyophilized to address aggregation issues when they are combined with the diluent to form the reagent.

Systems and methods disclosed herein are used for biological sample imaging using artificial particle references. The system includes a microscopy device, a diluent comprising fluorescent dyes, a sample holder, and a plurality of artificial particles. The artificial particles may be included in the liquid suspension, which may be lyophilized. The artificial particles and the biological sample are mixed with the diluent in the sample holder to form a solution. The biological sample is imaged with the artificial particle as reference markers using the microscopy device. The settling time of the artificial particles is shorter than or equal to the settling time of the biological sample.

Throughout the disclosure, the reagents used for the methods and systems disclosed refer to a mixture of liquid diluent and cakes within. The cakes refer to powders of dried products that once reconstituted support biological imaging purposes. The cakes may include, without limitations, artificial particles such as quality control (QC) beads, fluorescent stains, or/and a selection of chemicals including salts, surfactants, cell stabilizers, and other desirable chemicals. The liquid suspension may be lyophilized to form lyophilized cakes through a freezing drying technology. The liquid diluent may be, without limitations, water-based, phosphate-buffered saline (PBS), or any diluent with a specific function used to provide a desirable environment for biological imaging, such as a balanced pH range to maintain the viability of the biological samples.

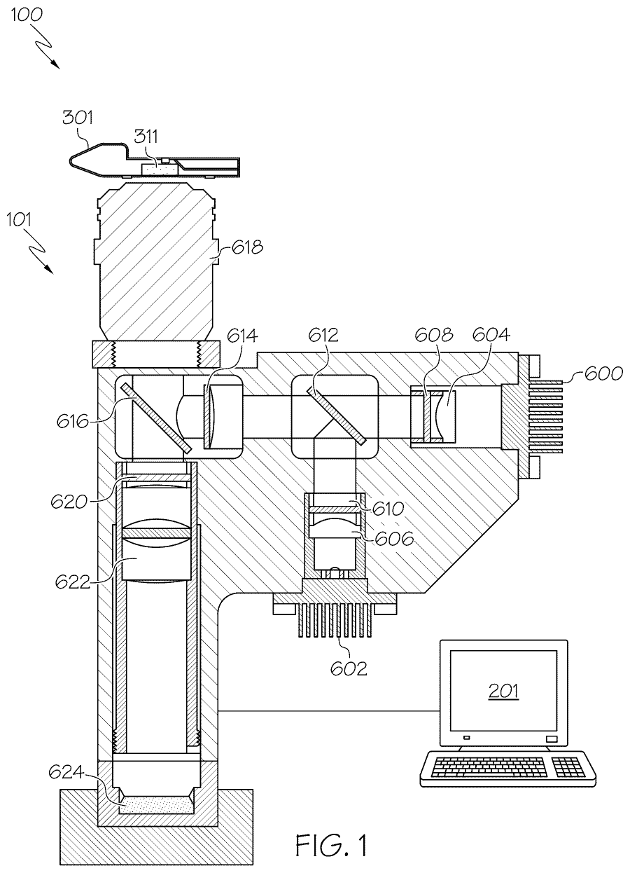

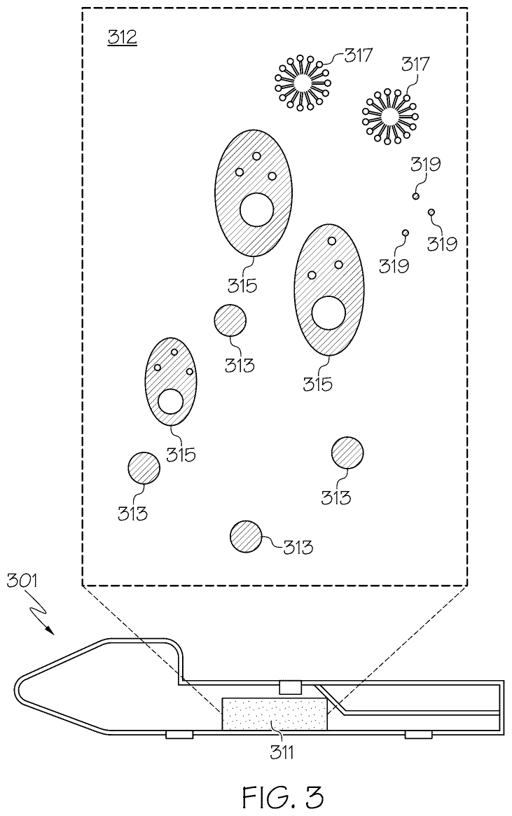

Turning now to figures, FIG. 1 depicts a biological imaging system 100. The system 100 includes a microscopy device 101 and a controller 201, which are communicatively coupled to each other through one or more connections. The biological imaging system 100 may further include a sample holder 301. The sample holder 301 may be, without limitations, a cartridge, a microslide, a lab-on-chip, or any containers for bio-microscopy imaging. In embodiments, the sample holder 301 may be a cartridge having a flat glass bottom and a top parallel to the glass bottom. The sample holder 301 may have several depths. The sample holder 301 may include a solution 311 and may be placed on top of the microscopy device 101 to be imaged. The solution 311, as further described in further below, may include a diluent 312, artificial particles 313, biological samples 315, uninterested particles 317, and dye 319 (e.g., as illustrated in FIG. 3). The solution 311 may further include cakes that are lyophilized and contain the artificial particles 313 such that the artificial particles do not aggregate in the solution 311.

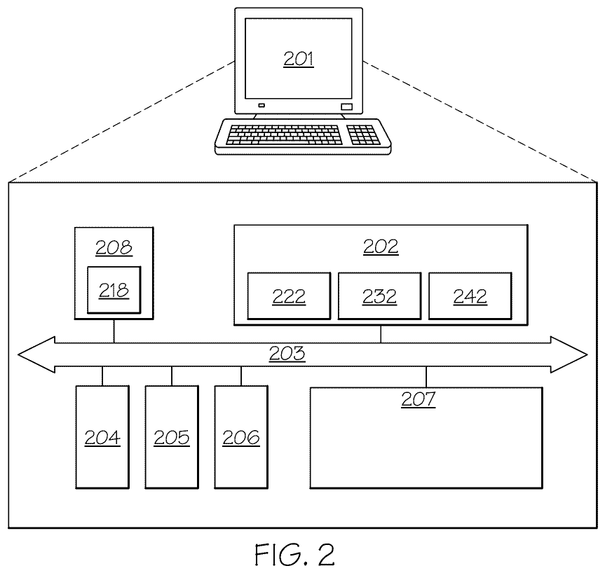

The biological imaging system 100 includes the controller 201, as shown in FIG. 2. The controller 201 includes an input/output interface 205 and the one or more connections. The one or more connections connect components of the biological imaging system 100 to the controller 201 and allow signal transmission between the components of the biological imaging system 100. For example, the connections may connect the microscopy device 101 via the connections. The connections may be formed from any medium that is capable of transmitting a signal such as, for example, conductive wires, conductive traces, optical waveguides, or the like. In some embodiments, the connections may facilitate the transmission of wireless signals, such as WiFi, Bluetooth®, Near Field Communication (NFC) and the like. Moreover, the connections may be formed from a combination of mediums capable of transmitting signals. In one embodiment, the connections may include a combination of conductive traces, conductive wires, connectors, and buses that cooperate to permit the transmission of electrical data signals to components such as processors, memories, sensors, input devices, output devices, and communication devices. Additionally, it is noted that the term “signal” means a waveform (e.g., electrical, optical, magnetic, mechanical or electromagnetic), such as DC, AC, sinusoidal-wave, triangular-wave, square-wave, vibration, and the like, capable of traveling through a medium. The controller 201 may receive inputs from the components and provide outputs to the components of the microscopy device 101.

The biological imaging system 100 depicted in FIG. 1 includes the microscopy device 101. The microscopy device 101 is, without limitations, an optical microscopy device, a light microscopy device, an electron microscopy device, a confocal microscopy device, a multiphoton microscopy device, or a fluorescence microscopy device. In some embodiments, the microscopy device 101 is a digital microscope. The microscopy device 101 may include, without limitations, one or more light sources, one or more lenses (such as, without limitations, a condenser lens and an objective lens), one or more mirrors and filters, one or more specimen stages, such as a well or a chamber, and any components and parts suitable for the operation of the microscopy device 101.

As illustrated in FIG. 1, the microscopy device 101 may include, without limitations, one or more light sources, including, without limitations, continuous spectrum light sources, multichromatic light sources, single-wavelength light sources, and fluorescence light sources of various wavelengths (such as a fluorescent blue light source 600 and a fluorescent ultraviolet light source 602), collector lenses 604 and 606, a blue excitation filter 608, an ultraviolet excitation filter 610, an excitation dichroic 612, a field lens 614, an imaging dichroic mirror 616, an objective lens 618, a triband filter 620, a tube lens 622, a camera 624. The microscopy device 101 is communicatively connected to the controller 201.

In operation, the microscopy device 101 may switch between, without limitations, a bright field mode, a dark field mode, and a fluorescent mode. In the bright field mode, the condenser lens (not shown in the figure) may focus and direct the light onto the solution 311 including one or more biological samples 315 or artificial particles 313 (FIG. 3), creating a bright and uniform background. The objective lens 618 may collect the transmitted light from the sample and form an image on the eyepiece or camera.

In the dark field mode, the condenser lens (not shown in the figure) may scatter or refract light to reach the objective lens 618, and the objective lens 618 may collect this scattered light and form an image of the biological samples 315 or the artificial particles 313 that appear bright against a dark background.

In the fluorescent mode, one or more excitation filters of different specific wavelengths, such as the blue excitation filter 608 and the ultraviolet excitation filter 610, may be used to select the excitation light that matches the absorption spectrum of the fluorophores in the solution 311. The imaging dichroic mirror 616 as a beamsplitter may reflect the excitation light towards the sample holder 301 while allowing emitted fluorescence to pass through and directed to the camera 624. The objective lens 618 may collect the emitted fluorescence from the biological samples 315 or the artificial particles 313 that are dyed with fluorescent stain or dye 319 (FIG. 3).

The sample holder 301 may be a cartridge, a microslide, or a lab-on-chip. In operation, the sample holder 301 including the biological sample 315 and the artificial particles 313 (FIG. 3) in the solution 311 may be placed in a well or a chamber (not illustrated) of the microscopy device 101 such that the solution 311 is placed along the path of the fluorescent blue light source 600 and/or the fluorescent ultraviolet light source 602. In particular, a biological sample 315 may be analyzed by the microscopy device 101, such that the results from the microscopy device 101 may be used to analyze the biological sample 315. One of the light sources, such as the fluorescent blue light source 600 or the fluorescent ultraviolet light source 602, illuminates the biological samples 315 or the artificial particles 313. An image of the biological samples 315 and/or the artificial particles 313 is captured by the camera 624. The image captured by the camera 624 may be transmitted to the controller 201 for automated analysis. An image and additional information may be displayed at a display 208 including a user interface 218 (FIG. 2). The microscopy device 101 may utilize microscopy techniques to determine attributes associated with sample preparation, reagent preparation, and biological analysis process. In the illustrated example, the attributes may be used to determine, without limitations, the settlement of the biological samples 315, a confirmation of the reagent process workflow, a confirmation of acellular sample in the solution, a confirmation of magnification of the microscopy device 101, and estimation of incubation time. However, in other examples, the microscopy device 101 may identify other attributes.

Referring to FIG. 2, example non-limiting components of the biological imaging system 100 are depicted. The biological imaging system 100 may include the controller 201. The controller 201 may include various components, such as a memory component 202, a processor 204, an input/output interface 205, a network interface hardware 206, a data storage component 207, a display 208 including a user interface 218, and a local interface 203. The controller 201 may include one or more displays 208 with one or more user interfaces 218. The controller 201 may include one or more modules, such as an automatic focusing module 222 to conduct real-time sequence focusing at various depths of the solution 311 (FIGS. 1, 3, and 4), an artificial particle module 232 to identify artificial particles 313 (FIG. 3), and an biological sample module 242 to identify biological samples (FIG. 3).

The controller 201 may be any device or combination of components comprising the processor 204 and the memory component 202, such as a non-transitory computer readable memory. The processor 204 may be any device capable of executing the machine-readable instruction set stored in the non-transitory computer readable memory. Accordingly, the processor 204 may be an electric controller, an integrated circuit, a microchip, a computer, or any other computing device. The processor 204 may include any processing component(s) configured to receive and execute programming instructions (such as from the data storage component 207 and/or the memory component 202). The instructions may be in the form of a machine-readable instruction set stored in the data storage component 207 and/or the memory component 202. The processor 204 is communicatively coupled to the other components of the controller 201 by the local interface 203. Accordingly, the local interface 203 may communicatively couple any number of processors 204 with one another, and allow the components coupled to the local interface 203 to operate in a distributed computing environment. The local interface 203 may be implemented as a bus or other interface to facilitate communication among the components of the controller 201. While the embodiment depicted in FIG. 2 includes a single processor, other embodiments may include more than one processor.

The memory component 202 (e.g., a non-transitory computer-readable memory component) may include RAM, ROM, flash memories, hard drives, or any non-transitory memory device capable of storing machine-readable instructions such that the machine-readable instructions can be accessed and executed by the processor 204. The machine-readable instruction set may include logic or algorithm(s) written in any programming language of any generation (e.g., 1GL, 2GL, 3GL, 4GL, or 5GL) such as, for example, machine language that may be directly executed by the processor 204, or assembly language, object-oriented programming (OOP), scripting languages, microcode, etc., that may be compiled or assembled into machine readable instructions and stored in the memory component 202. Alternatively, the machine-readable instruction set may be written in a hardware description language (HDL), such as logic implemented via either a field-programmable gate array (FPGA) configuration or an application-specific integrated circuit (ASIC), or their equivalents. Accordingly, the functionality described herein may be implemented in any conventional computer programming language, as pre-programmed hardware elements, or as a combination of hardware and software components. For example, the memory component 202 may be a machine-readable memory (which may also be referred to as a non-transitory processor-readable memory or medium) that stores instructions that, when executed by the processor 204, causes the processor 204 to perform a method or control scheme as described herein. While the embodiment depicted in FIG. 2 includes a single non-transitory computer-readable memory component, other embodiments may include more than one memory module. The memory component 202 may be used to store the one or more modules. The one or more modules during operating may be in the form of operating systems, application program modules, and other program modules. Such program modules may include, but are not limited to, routines, subroutines, programs, objects, components, and data structures for performing specific tasks or executing specific abstract data types according to the present disclosure as will be described below. For example, the program module may include the automatic focusing module 222 to conduct real-time sequence focusing at various depths of the solution 311 (FIGS. 1, 3, and 4), the artificial particle module 232 to identify artificial particles 313 (FIG. 3), the biological sample module 242 to identify biological samples (FIG. 3).

The input/output interface 205 may include a monitor, keyboard, mouse, printer, camera, microphone, speaker, and/or other device for receiving, sending, and/or presenting data. The network interface hardware 206 may include any wired or wireless networking hardware, such as a modem, LAN port, Wi-Fi card, WiMax card, mobile communications hardware, and/or other hardware for communicating with other networks and/or devices. The data storage component 207 may store the one or more modules. The input/output interface 205 and the network interface hardware 206 allow a user to send input to the controller 201 of the biological imaging system 100 to control and manipulate the components of the biological imaging system 100, such as the fluorescent blue light source 600, the fluorescent ultraviolet light source 602, the collector lenses 604 and 606, the blue excitation filter 608, the ultraviolet excitation filter 610, the excitation dichroic 612, the field lens 614, the imaging dichroic mirror 616, the objective lens 618, the triband filter 620, the tube lens 622, and the camera 624, and receive output from the controller 201.

In operation, the camera 624 may capture image data and communicates the image data to the processor 204. The image data may be received by the processor 204, which may process the image data using one or more image processing algorithms. Any known or yet-to-be developed video and image processing algorithms may be applied to the image data in order to identify an item or situation. Example video and image processing algorithms include, but are not limited to, kernel-based tracking (such as, for example, mean-shift tracking) and contour processing algorithms. In general, video and image processing algorithms may detect objects and movement from sequential or individual frames of image data. One or more object recognition algorithms may be applied to the image data to extract objects and determine their relative locations to each other. Any known or yet-to-be-developed object recognition algorithms may be used to extract the objects or even optical characters and images from the image data. Example object recognition algorithms include, but are not limited to, scale-invariant feature transform (“SIFT”), speeded up robust features (“SURF”), and edge-detection algorithms.

The data storage component 207 stores collected imaging data and data of operating various components of the microscopy device 101. The various modules may also be stored in the data storage component 207 during operating or after operation.

The display 208 include a user interface 218, a screen, one or more devices in communication with the one or more processors 204 (such as smartphones, tables, and the like), and/or any other device or interface suitable for displaying data. In some examples, the display 208 is a touchscreen and may be configured as an input device to receive user input.

Each of the various modules may include one or more machine learning algorithms or neural networks. Each module may be trained and provided machine learning capabilities via a neural network as described herein. By way of example, and not as a limitation, the neural network may utilize one or more artificial neural networks (ANNs). In ANNs, connections between nodes may form a directed acyclic graph (DAG). ANNs may include node inputs, one or more hidden activation layers, and node outputs, and may be utilized with activation functions in the one or more hidden activation layers such as a linear function, a step function, logistic (sigmoid) function, a tanh function, a rectified linear unit (ReLu) function, or combinations thereof. ANNs are trained by applying such activation functions to training data sets to determine an optimized solution from adjustable weights and biases applied to nodes within the hidden activation layers to generate one or more outputs as the optimized solution with a minimized error. In machine learning applications, new inputs may be provided (such as the generated one or more outputs) to the ANN model as training data to continue to improve accuracy and minimize error of the ANN model. The one or more ANN models may utilize one to one, one to many, many to one, and/or many to many (e.g., sequence to sequence) sequence modeling. The one or more ANN models may employ a combination of artificial intelligence techniques, such as, but not limited to, Deep Learning, Random Forest Classifiers, Feature extraction from audio, images, clustering algorithms, or combinations thereof. In some embodiments, a convolutional neural network (CNN) may be utilized. For example, a CNN may be used as an ANN that, in a field of machine learning, for example, is a class of deep, feed-forward ANNs applied for audio analysis of the recordings. CNNs may be shift or space invariant and utilize shared-weight architecture and translation. Further, each of the various modules may include generative artificial intelligence algorithms. The generative artificial intelligence algorithm may include a general adversarial network (GAN) that has two networks, a generator model and a discriminator model. The generative artificial intelligence algorithm may also be based on variation autoencoder (VAE) or transformer-based models.

In some embodiments, the controller 201 may connect to a server or other systems, such as Internet of Things (IoTs) through the network interface hardware 206 to send acquired imaging data for further analysis and receive recommendations for further operation of the system 100. The system 100 may monitor the operation of the microscopy device 101 including the focusing performance and acquired image data and transfer real-time and/or recorded data (such as videos, images, messages, status, warning, and instructions, etc.) to a server or a user via wired or wireless connections, such as, without limitations, a cellular connection, internet, and any radio-wave based communication.

Referring to FIG. 3, the example solution 311 in the sample holder 301 is depicted. The solution 311 is a mixture with the combination of biological samples 315 and the artificial particles 313 in the diluent 312. In some embodiments, the solution 311 may further include dye 319 and impurities, such as uninterested particles 317, such as, without limitations, lipids, pollen of the biological samples 315, grass, dirt, dust, or talc (e.g. from gloves).

The biological samples 315 may include cells and/or tissues of humans, animals, and/or plants, or other organic materials derived from living organisms, such as, without limitations, organisms (such as, without limitations, bacteria, yeast, mites, parasites, worms, microfilaria, babesia, and malaria), microbes, tissue, organelles, and macromolecular assemblies inside the cell. The use of the system 100 may provide the user to visualize the dynamics of biological sample 315, individual organelles, and macromolecular assemblies inside the cell. The system 100 may provide information for studying the location of signaling pathways and binding partners.

The artificial particles 313 are non-naturally occurring materials. The artificial particles 313 may include, without limitations, one or more of polymeric beads or inorganic beads, wherein the polymeric beads are, without limitations, polystyrene beads, acrylic beads, polyethylene beads, polypropylene beads, polyacrylate beads, or Polymethylhydrosiloxane (PMHS), and the inorganic beads are, without limitations, glass beads, silicon dioxide beads, or metal beads. The artificial particles 313 may have various controlled surface morphology. The surface morphology may include, without limitations, a spherical shape, a golf-ball shape, or a popped-corn shape. The artificial particles 313 may be functionalized beads with one or more functional groups. The functional groups may be selected from, without limitations, carboxyl, hydroxyl, amine, enzymes, antibodies, aptamers, or a combination thereof. In some embodiments, the artificial particles 313 may be used for, without limitations, biomolecule separation, protein purification, immunoassays, drug delivery systems, and diagnostic tests. The artificial particles 313 may include functionality for precise interactions with biological molecules, materials, and nanoparticles, enabling targeted or desired outcomes.

The dye 319 may be fluorescent that emits light when exposed to specific wavelengths of light. The dye 319 may be used in biological imaging to provide brightness and photostability by labeling the cells, especially the live cells. The dye 319 may also be used for quantification purpose to provide concentration or relative abundance information of interested biological samples in association with the intensity of the dye signals. The dye 319 may be used to detect proteins and nucleic acids. The dye 319 may be, without limitations, cyanine dye, boron dipyrromethene (BODIPY) dye, near-infrared dyes, Janelia Fluor (JF) dyes, Phthalocyanines, Squaraine dyes, Perylene diimide (PDI), or a combination thereof. The dye 319 may be attached to the biological sample 315 as tags to antibodies that attach to the biological sample 315, or by staining in a specific manner.

The diluent 312 is a liquid or a solution used to dilute or reduce the concentration of a sample or reagent. The diluent 312 may be used to create a more suitable environment for imaging processes, ensuring that the sample is at the right concentration and conditions for accurate analysis. The diluent 312 may be a water-based solution, a phosphate-buffered saline (PBS) based solution, a tris-buffered saline (TBS) based solution, or a salt-based solution. The diluent 312 may provide a stable pH level and appropriate ionic strength for the biological sample 315. The diluent 312 may also provide nutrients and maintain the physiological conditions required for cell survival during imaging.

The impurities or the uninterested particles 317 may be introduced during the process of preparation of the solutions or from the components of the solutions, such as, without limitations, lipids, pollen, grass, dirt, dust, or talc (e.g. from gloves), that are separate and do not dissolve in the solution 311. The modules of the system 100, such as the biological sample module 242 and the artificial particle module 232, may include algorithms to identify the impurities or the uninterested particles 317. In some embodiments, the system 100 may detect and image the impurities and the uninterested particles 317 under the request of the user.

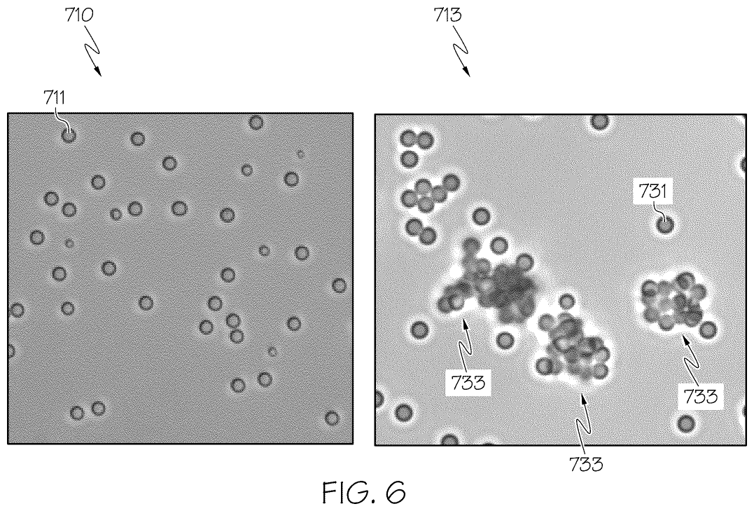

In embodiments, the artificial particles 313 may be included in the lyophilized cakes that include fluorescent stains, or/and a selection of chemicals including salts, surfactants, cell stabilizers, and other desirable chemicals. The cakes may be, as described in detail further below, formed after lyophilized drying such that the artificial particles 313 do not aggregate in the solution (FIG. 6). In some embodiments, the cakes may be included in the reagent before being added to the diluent 312 to form the solution 311. In some embodiments, the cakes including the artificial particles 313 and the dyes 319 may be directly added to the diluent 312 to form the solution 311, without a step of forming the reagent.

In the solution 311, the dyes 319 may react with the biological sample 315 or the artificial particles 313 to render the biological sample 315 or the artificial particles 313 fluorescent. In some embodiments, the artificial particles 313 may include a functionalized surface of functional ligands that attract selective dyes 319 that match the ligands. In some embodiments, the dyes 319 are attached to selective antibodies that are attached to certain types of cells within the biological sample 315. The selective binding and/or attraction between functional ligands on the artificial particles 313 to the corresponding dyes 319, and the antibodies to the certain cells enhances the specificity of the labeling or fluorescence process.

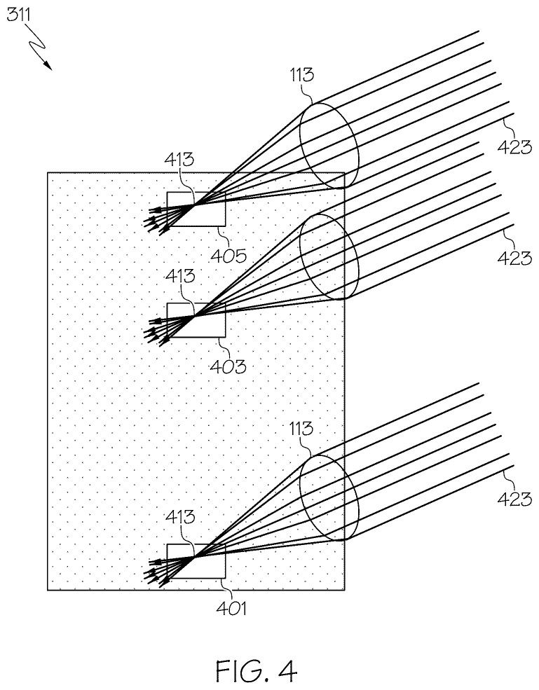

Referring to FIG. 4, various imaging modes with the artificial particle 313 as references are depicted. After the sample holder 301 is placed in the well or chamber of the microscopy device 101, the system 100 may focus, using the automatic focusing module 222 (FIG. 2) at different depths of the solution 311 to detect particles of interest. The automatic focusing module 222 position the focal point 413 of the microscopy device 101 by manipulating the objective lens 113 to refract incoming light 423 and achieve focus at one or more specified depths of interest. The focal point 413 can be controlled to shift within a region of interest, such as near the surface area 405 of the solution 311, within a region 403 expected to contain desired particles, or within the lowermost region 401 of the solution 311. The system 100 may capture one or more in-focus images containing the artificial particles 313. The system 100 can detect biological samples 315 or uninterested particles 317 at various depths of interest. It then utilizes the artificial particle module 232 and/or the biological sample module 242 to analyze the images. This analysis informs further actions for the microscopy device 101, and it also communicates messages to the user through the user interface 218. These messages pertain to various system tasks including, but not limited to, confirming the settlement of the biological sample 315, verifying the presence of reagents, confirming the presence of acellular samples within the biological sample 315, and confirming the current magnification level of the microscopy device 101.

The artificial particles 313 may have uniform sizes, with a deviation distribution below a threshold deviation value. The artificial particles 313 may have a uniform size or two uniform sizes. For example, the diameters of the artificial particles 313 may have a nominal size of 5 microns with a size range of between 1 micron and 10 microns and a standard deviation of less than 1.5 microns. The artificial particles 313 may have a size less than the biological sample 315. The artificial particles 313 may have a density greater than the diluent 312 and the reagent. In some embodiments, metal or magnetic elements may be added to the artificial particles 313 to increase the density of the artificial particles 313 or otherwise provide an alternate means to accelerate the settling of the artificial particles 313.

The artificial particles 313 and the biological samples 315 may have a density lower than or greater than the solution 311 or the diluent 312. In some embodiments, when the densities of the artificial particles 313 and the biological samples 315 are greater than those of the solution 311 or the diluent 312, the artificial particles 313 and the biological samples 315 may settle to the bottom of the solution 311. During the focus sequences of the method for use with a biological sample 315, the settlement of the artificial particles 313 or the biological sample 315 may be determined by arranging the focal point 413 in depths around the lowermost region 401 of the solution 311 and capturing an in-focus image of the artificial particles 313 or the biological sample 315. In some embodiments, when the densities of the artificial particles 313 and the biological samples 315 are lower than the densities of the solution 311 or the diluent 312, the artificial particles 313 and the biological samples 315 may settle to the top of the solution 311. The focal point 413 may be arranged in depths around the surface area 405 of the solution 311 to capture an in-focus image of the artificial particles 313 or the biological sample 315.

The settling rate and settling time of the artificial particles 313 are relevant to the size and density of the artificial particles. Generally, the settling rate of the artificial particles 313, the biological sample 315 such as cells, and the uninterested particles 317 depend on their size, shape, density, and the specific gravity and viscosity of the solution 311 where the particles/cells are. The settling time may be further calculated based on the settling rates of the particle/sample, the initial place of the particle/sample in the sample holder 301, and the size of the sample holder 301. The system 100 can incorporate an algorithm designed to calculate the settling rates and settling times of both the artificial particles 313 and the biological sample 315 using principles such as Stokes' law or Newton's law. Additionally, this algorithm can be employed to determine the location within the sample holder 301 where the artificial particles 313 and the biological sample 315 are to be found, such as near the surface area 405 of the solution 311, within a region 403 expected to contain desired particles, or within the lowermost region 401 of the solution 311. In some embodiments, the artificial particles 313 settle faster than the biological sample 315, up to four times as fast depending on the size of the particle/cell.

In embodiments, the settling time of the artificial particles 313 may be greater than or equal to 5 seconds and less than or equal to 200 seconds. The settling rate of the artificial particles 313 may be greater than or equal to 1 micron per second and less than or equal to 1000 microns per second. The settling time of the artificial particles 313 may be a function of the depth of the sample holder 301 and the settling rate of the artificial particles 313.

In embodiments, the system 100 may first determine whether artificial particles 313 are detected at one or more of the scanned depths of interest. After the detection of the artificial particles 313 at certain depths, the system 100 further to determine whether the biological sample 315 or other particle of interest, or any dynamic changes of the biological sample 315 is present at the one or more depths of interest. The system 100 then captures an in-focus image of the biological sample 315 or other interested particles after an affirmative determination of their presence and use various algorithms to determine whether one or more imaging events, such as, without limitation, confirmation of the settlement of the biological sample 315, a confirmation of reagent, a confirmation of acellular sample contained in the biological sample 315, a confirmation of magnification of the microscopy device 101, or estimation of incubation time of the biological sample 315, the fluorescent dye 319, or the artificial particles 313, are present and provide corresponding information at the user interface 218.

The artificial particles 313 can be used for magnification reference. The microscopy device 101 may include a magnification based on the parameters of the components of the microscopy device 101. After detecting the artificial particles 313 in the solution, which include known particle sizes with a controlled size variation, the system 100 may use the size of the artificial particle 313 as a reference to verify the system-based magnification. The magnification verification may be conducted when optical artifacts, such as, without limitations, halos around cells, are present to use the artificial particles 313 around the biological samples 315 for each run to confirm the size calculations of the biological samples 315 are correct.

The artificial particles 313 can be used for fluorescence signal reference. Fluorescence signals may change due to several factors within the optics and within the reagents. The artificial particles 313 may be fluorescent and may be used to determine the dynamics of the fluorescence signals in the reagents. For example, the artificial particles 313 may be chemically altered to respond to the fluorescent stains and fluoresce in a controlled manner. The controller chemical changes of the artificial particles may be used by one or more algorithms of the system 100 to interpret the kinetics of fluorescent signals of the biological samples 315. This method can also provide information about the efficacy of the stain. The stain may degrade over time or/and due to thermal or light exposure).

The artificial particles 313 can be used for confirmation of the biological samples 315 that include acellular samples. For example, sample collection for certain cytology may result in no cells being aspirated into needles used to collect the biological sample 315. In embodiments, the system 100 may select one or more areas of interest 503 and conduct sequence focus using the automatic focusing module to determine whether acellular samples are present in the solution 311.

With the artificial particles 313 as imaging references in the solution 311, the system 100 may focus on an area of interest 503 in the solution 311 to search for the expected presence of biological samples 315 and cells during a pre-scan in the sample holder 301 placed in the well or the chamber of the microscopy device 101. The pre-scan step may be conducted during a biological sample or cells distribution step before the biological samples 315 are settled in the solution 311. The system 100 may include a predetermined concentration of the artificial particles 313 for a confirmation that more than a threshold amount of artificial particles 313 are present in the solution but less than a threshold amount or concentration of biological samples 315 are found in the solution 311. The threshold concentration of biological sample 315 is a minimum value to secure the presence of biological sample 315. The threshold value of the biological sample 315 may be predetermined based on the type and size of the biological sample 315 and may evolve as more data are collected during the usage of the system 100 using a machine learning algorithm associated with the biological sample module.

The artificial particles 313 can be used for confirmation of reagent steps. In embodiments, various reagent steps are performed before the solution 311 is formed. For example, the user may dispense the biological sample 315 into the diluent 312. This diluent 312 may temporarily preserve the cells in the biological sample 315. The user may further add the lyophilized reagents, which includes fluorescent dyes 319 and the artificial particles 313, to the dilute 312. The lyophilized reagents may dissolve in the diluent 312 within a few seconds and be agitated to form a uniformly distributed solution 311. The mixed solution 311 may then be transmitted into the sample holder 301 for further analysis.

When a user misoperates or skips some of the reagent steps such as, for example, the lyophilized reagents may not be dissolved in the diluent 312 or flow into the sample holder 301, the system 100 may conduct a pre-scan step using the automatic focusing module 222 and the artificial particle module 232 to determine whether the reagent steps are conducted properly and whether the solution 311 is satisfied for further characterization. For example, the pre-scanning under the fluorescence mode may detect a lack of presence of the fluorescent dyes suggesting that a lyophilization step is missed, or an absence of the artificial particles 313 indicating that the sample steps may not have been followed correctly. The system 100 then displays a corresponding error message at the user interface 218 and may further provide reasons of the error(s) and instruct further steps to correct the errors.

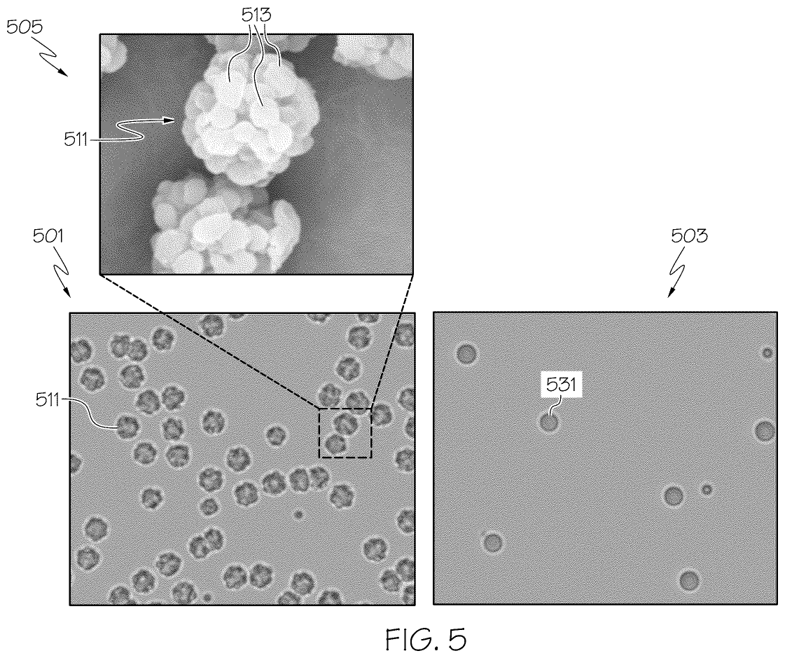

Referring to FIG. 5, examples of artificial particles 313 with different surface morphologies are depicted. In embodiments, the artificial particles 313 may be polymer beads. The artificial particles 313 may have refractive index values similar to blood cells. The artificial particles 313 may have similar sizes (e.g. 2-20 microns or 4-6 microns to represent red blood cells). In some embodiments, as illustrated in 503 of FIG. 5, the artificial particles 313 may be in a spherical shape 531 and stable at room temperature for a long period, such as 1-10 years. In some embodiments, as illustrated in image 501 of FIG. 5, the artificial particles 313 are further cross-polymerized via cross-linking which changes the spherical nature of the artificial particles 313 to a golf ball shape or popcorn shape 511. The impact of the cross-linking is that the spherical artificial particles 531 form a series of larger artificial particles of the popcorn shape particles 511 that are made up of smaller spheres 513, as shown in image 505 in FIG. 5. The morphology of the artificial particles 313, such as the spherical shape, or the golf ball shape or popped corn shape, may be used by the modules, such as the artificial particle module 232 and the biological sample module 242 to identify the artificial particles 313 from the biological sample 315, such as natural cells.

In embodiments, both the artificial particles 313 having the spherical shape 531 and the popcorn shape 511 can illuminate with specific brightfield wavelengths that allow the system 100 to differentiate the artificial particles 313 from the biological sample 315 and the uninterested particles 317. Moreover, in cases where the artificial particles 313 are not labeled with fluorescent dye 319, the system 100 may be capable of distinguishing between the biological sample 315, which may contain RNA and/or DNA and is stained with either brightfield or fluorescent stains, and the artificial particles 313 that remain unaffected by these stains. The disclosed method can be used to detect Red Blood Cells (RBC) such that an image captured using brightfield microscopy using violet light, green light, or other lights can find artificial particles 313 in bright color and RBC in dark color.

In some embodiments, the artificial particle module 232 and the biological sample module 242 may include an algorithm to identify and differentiate the artificial particles 313 and the biological sample 315. The algorithm may adopt one or more offsets in differentiating the artificial particles 313 and the biological sample 315 based on the size, the fluorescent wavelength, or the surface morphology between the artificial particles 313 and the biological sample 315. For example, the size of the artificial particles 313 may be more than 10 microns smaller than the biological sample, include no fluorescent activated light, and have a popcorn-shape morphology, while the biological sample 315 may be at least 10 microns larger in diameter, be dyed to emit fluorescent activated light, and have a typical cell shape, such as spherical, oval, elliptical, spindle shape, polygonal, isodiametric, or flat plate-like.

Referring to FIG. 6, distributions of artificial particles and other particles in the liquid suspension with and without lyophilization treatment in the solution are depicted. As shown in image 710 of FIG. 6, the spherical particles 711, which include the artificial particles 313, are separate after the lyophilized cake dissolved in the diluent 312. The artificial particles 313 are not aggregated as a cluster 733 as shown in image 713. In contrast, as illustrated in image 713 of the FIG. 6, the spherical particles 731, which include the artificial particles 313, may partially aggregate to form a cluster 733 after being resuspended in the diluent 312. In operation, the non-aggregate particle 313 in the cakes are lyophilized under one or more freezing cycles and one or more drying cycles, wherein temperatures of the freezing cycles are greater than or equal to −50° C. and less than or equal to 5° C., and temperatures of the drying cycles are greater than or equal to −50° C. and less than or equal to 25° C.

In some embodiments, the components contained in the cakes are prepared in the form of bulk reagent and are pretreated before lyophilization. The bulk reagent in a tube may be inverted several times to keep the particles well-suspended. The resuspended bulk reagent may be dispensed into a glass vial or plastic tube. All the glass vials may be placed in a metal tray and the tray may be placed in a lyophilizer. Sample lyophilization cycles are provided below. In the sample lyophilization cycles, there are four freezing cycles and nine drying cycles. The freezing cycles are conducted under temperatures between −50° C. to 5° C., for 0 min to 200 min. The drying cycles are conducted under temperatures between −50° C. to 25° C., at pressure of 40 mTorr, for 0 to 1300 min.

| Freezing | 1st | 2nd | 3rd | 4th |
|---|---|---|---|---|
| cycle | cycle | cycle | cycle | cycle |
| Temp. (° C.) | 5 | 5 | −50 | −50 |
| Time (min) | 0 | 30 | 0 | 200 |

| Drying | 1st | 2nd | 3rd | 4th | 5th | 6th | 7th | 8th | 9th |
|---|---|---|---|---|---|---|---|---|---|
| cycle | cycle | cycle | cycle | cycle | cycle | cycle | cycle | cycle | cycle |
| Temp (° C.) | −50 | −33 | −33 | −20 | −20 | −10 | −10 | 25 | 25 |
| Time (min) | 0 | 65 | 1300 | 65 | 700 | 40 | 100 | 125 | 500 |
| Pressure | 40 | 40 | 40 | 40 | 40 | 40 | 40 | 40 | 40 |
| (mTorr) | | | | | | | | | |

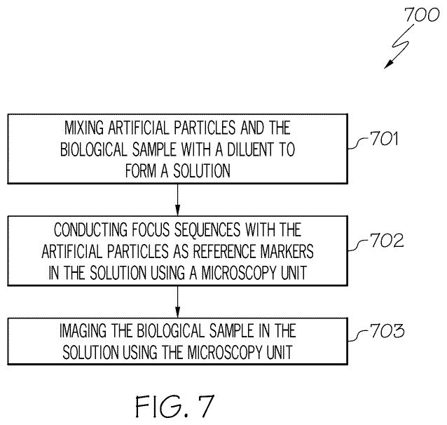

Referring to FIG. 7, a flowchart of an example method 700 of biological sample imaging using the biological optical imaging system 101 is depicted, with reference to FIGS. 3-4. At step 701, the method for use with a biological sample 315 includes mixing artificial particles 313 and the biological sample 315 with a diluent 312 to form a solution 311. The artificial particles 313 are suspended in the solution 311 independent from the biological sample 315. A settling time of artificial particles 313 is shorter than or equal to a settling time of the biological sample 315. At step 702, the method for use with a biological sample 315 includes, conducting focus sequences with the artificial particles 313 as reference markers in the solution 311 using a microscopy device. At step 703, the method for use with a biological sample includes imaging the biological sample in the solution using the microscopy device.

In some embodiments, the method 700 may include, after mixing to form the solution, dispensing the solution into the sample holder 301, such as a cartridge used by the microscopy unit.

In some embodiments, the method 700 may include an imaging algorithm that adopts one or more offsets, based on a difference between the artificial particle 313 and the biological sample 315 in size, fluorescent wavelength, or surface morphology, to identify the artificial particles and the biological sample. The size of the artificial particles 313 may be, without limitations, greater than or equal to 1 micron and less than or equal to 20 microns. The artificial particles 313 may have the surface morphology of, without limitations, a spherical shape, a golf-ball shape, or a popped-corn shape. The biological sample 315 may include a fluorescent stain.

In some embodiments, the mixing may further include a reagent process. The reagent process may include dispensing the biological sample 315 into a sample holder 301 containing the diluent 312 including the fluorescent stain 319, adding lyophilized cakes into the sample holder 301, and agitating the sample holder 301 to mix the biological sample 315, the lyophilized cakes that may include the artificial particles 313 and the dye 319, and the diluent 312. In some embodiments, the biological sample 315 may be dyed with the dye 319 that may be fluorescent stain during the reagent process.

In some embodiments, the method 700 may include arranging a focal point 413 of the microscopy device 101 in one or more depths of interest, such as near the surface area 405 of the solution 311, within a region 403 expected to contain desired particles, or within the lowermost region 401 of the solution 311. The method 700 may further include capturing an in-focus image of the artificial particles 313 at the one or more depths of interest. The method may include in response to capturing the in-focus image of the artificial particles 313, determining whether the biological sample 315 is present at the one or more depths of interest. The method 700 may include, in response to the determination of the biological sample 315 at the one or more depths of interest, capturing an in-focus image of the biological sample 315 to determine one or more imaging events, the imaging events may be, without limitation, a settlement of the biological sample, a confirmation of reagent, a confirmation of acellular sample, a confirmation of magnification, or an estimation of incubation time.

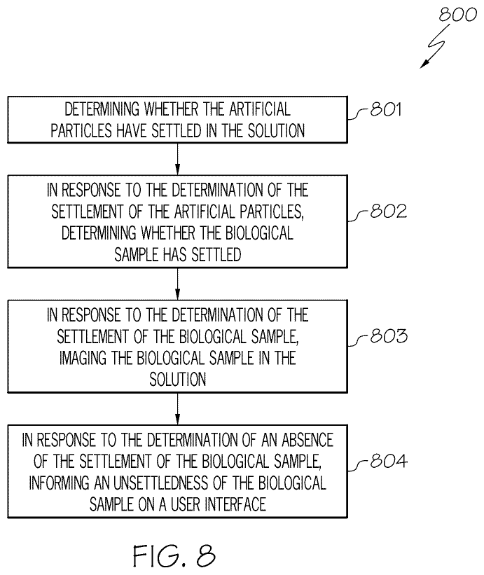

Referring to FIG. 8, a flowchart of an example method 800 of focus sequences using the biological optical imaging system is depicted, with reference to FIGS. 3-4. In embodiments, at step 801, the focus sequences of the method of biological sample imaging may include determining whether the artificial particles 313 have settled in the solution 311. At step 802, in response to the determination of the settlement of the artificial particles 313, the focus sequences may include determining whether the biological sample 315 has settled. At step 803, in response to the determination of the settlement of the biological sample 315, the focus sequences may include imaging the biological sample 315 in the solution. At step 804, in response to the determination of an absence of the settlement of the biological sample 315, the focus sequences may include providing an unsettledness of the biological sample on a user interface.

In some embodiments, the focus sequences may further include providing a reason for the unsettledness of the biological sample on the user interface, wherein the reason is associated with an error message of a pre-analytical step. The artificial particles 313 and the biological samples 315 may have a density lower than or greater than the solution 311 or the diluent 312. In some embodiments, when the density of the artificial particles 313 and the biological samples 315 are greater than the density of the solution 311 or the diluent 312, the artificial particles 313 and the biological samples 315 may settle to the bottom of the solution 311. During the focus sequences of the method for use with a biological sample 315, the settlement of the artificial particles 313 or the biological sample 315 may be determined by arranging a focal point 413 of the microscopy device 101 in a depth around the bottom 401 of the solution 311 and capturing an in-focus image of the artificial particles 313 or the biological sample 315. In some embodiments, when the density of the artificial particles 313 and the biological samples 315 are less than the density of the solution 311 or the diluent 312, the artificial particles 313 and the biological samples 315 may settle to the top of the solution 311. During the focus sequences of the method for use with a biological sample, the settlement of the artificial particles 313 or the biological sample 315 may be determined by arranging a focal point 413 of the microscopy device 101 in a depth around top 413 of the solution 311, and capturing an in-focus image of the artificial particles or the biological sample around the top 413 of the solution 311.

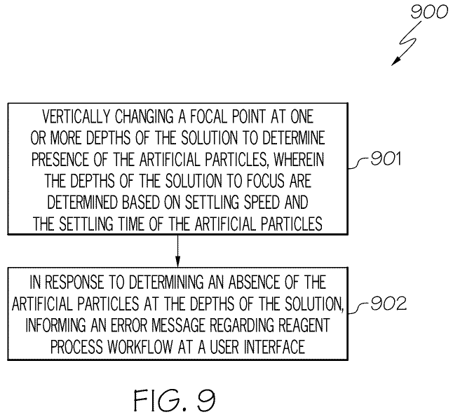

Referring to FIG. 9, a flowchart of an example method 900 of confirmation of reagents using the biological optical imaging system is depicted, with reference to FIGS. 3-4. At step 901, the method 900 of confirmation of reagents may include vertically changing a focal point 413 at one or more depths of the solution 311 to determine presence of the artificial particles 313. The depths of the solution to focus may be determined based on the settling speed and the settling time of the artificial particles 313. At step 902, in response to determining an absence of the artificial particles 313 at the depths of the solution, the method 900 of confirmation of reagents using the biological optical imaging system 100 may further include providing an error message regarding reagent process workflow at a user interface 218.

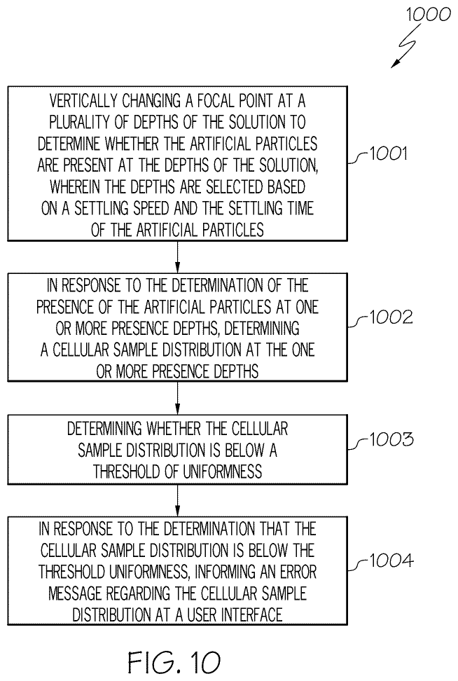

Referring to FIG. 10, a flowchart of an example method 1000 of confirmation of acellular using the biological optical imaging system is depicted, with reference to FIGS. 3-4. At step 1001, the method 1000 of confirmation of acellular using the biological optical imaging system 100 may include vertically changing a focal point 413 at a plurality of depths of the solution 311 to determine whether the artificial particles 313 are present at the depths of the solution 311, wherein the depths are selected based on a settling speed and the settling time of the artificial particles 311. At step 1002, the method 1000 of confirmation of acellular using the biological optical imaging system 100 may include, in response to the determination of the presence of the artificial particles 313 at one or more presence depths, determine a cellular sample distribution at the one or more presence depths. At step 1003, the method 1000 of confirmation of acellular using the biological optical imaging system 100 may include determining whether the cellular sample distribution is below a threshold density. At step 1004, the method 1000 of confirmation of acellular using the biological optical imaging system 100 may include, in response to the determination that the cellular sample distribution is below the threshold density, providing an error message regarding the cellular sample distribution at a user interface.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method for use with a biological sample, the method comprising:
mixing the biological sample and artificial particles with a diluent to form a solution;
conducting focus sequences with the artificial particles as reference markers in the solution using a microscopy device;
imaging the biological sample in the solution using the microscopy device;
wherein:
the artificial particles are suspended in the solution independent from the biological sample; and
a settling time of artificial particles is shorter than or equal to a settling time of the biological sample.

2. The method of claim 1, wherein the focus sequences comprise:
determining whether the artificial particles have settled in the solution;
in response to the determination of the settlement of the artificial particles, determining whether the biological sample has settled;
in response to the determination of the settlement of the biological sample, imaging the biological sample in the solution; and
in response to the determination of an absence of the settlement of the biological sample, providing an unsettledness of the biological sample on a user interface.

3. The method of claim 2, wherein the focus sequences further comprise providing a reason for the unsettledness of the biological sample on the user interface, wherein the reason is associated with an error message of a pre-analytical step.

4. The method of claim 2, wherein:
the settlement of the artificial particles or the biological sample is determined by arranging a focal point of the microscopy device in a depth around bottom of the solution, and capturing an in-focus image of the artificial particles or the biological sample.

5. The method of claim 2, wherein:
the settlement of the artificial particles or the biological sample is determined by arranging a focal point of the microscopy device in a depth around top of the solution, and capturing an in-focus image of the artificial particles or the biological sample around the top of the solution.

6. The method of claim 1, wherein:
the settling time of the artificial particles is greater than or equal to 5 seconds and is less than or equal to 200 seconds; and
a settling rate of the artificial particles is greater than or equal to 1 μm/s and less than or equal to 1000 μm/s.

7. The method of claim 1, wherein the artificial particles comprise one or more of polymeric beads or inorganic beads, wherein the polymeric beads are polystyrene beads, acrylic beads, polyethylene beads, polypropylene beads, polyacrylate beads, or Polymethylhydrosiloxane (PMHS), and the inorganic beads are glass beads, silicon dioxide beads, or metal beads.

8. The method of claim 1, wherein an imaging algorithm adopts one or more offsets, based on a difference between the artificial particles and the biological sample in size, fluorescent wavelength, or surface morphology, to identify the artificial particles and the biological sample.

9. The method of claim 8, wherein:
the size of the artificial particles is greater than or equal to 1 micron and less than or equal to 20 microns; and
the artificial particles have the surface morphology of a spherical shape, a golf-ball shape, or a popped-corn shape.

10. The method of claim 1, wherein the artificial particles are functionalized beads with one or more functional groups, wherein the functional groups are selected from carboxyl, hydroxyl, amine, enzymes, antibodies, aptamers, or a combination thereof.

11. The method of claim 1, wherein:
the artificial particles are included in cakes, wherein the cakes comprise fluorescent dyes;
the biological sample comprises a fluorescent stain; and
the cakes are lyophilized cakes formed under one or more freezing cycles and one or more drying cycles, wherein temperatures of the freezing cycles are greater than or equal to −50° C. and less than or equal to 5° C., and temperatures of the drying cycles are greater than or equal to −50° C. and less than or equal to 25° C.

12. The method of claim 1, wherein the mixing further comprises a reagent process, wherein the reagent process comprises:
dispensing the biological sample into a sample holder containing the diluent comprising a fluorescent stain;
adding lyophilized cakes into the sample holder; and
agitating the sample holder to mix the biological sample, the lyophilized cakes, and the diluent.

13. The method of claim 1, wherein the method further comprises:
vertically changing a focal point at one or more depths of the solution to determine presence of the artificial particles, wherein the depths of the solution to focus are determined based on settling speed and the settling time of the artificial particles; and
in response to determining an absence of the artificial particles at the depths of the solution, providing an error message regarding reagent process workflow at a user interface.

14. The method of claim 1, wherein the biological sample comprises cellular samples, the method further comprises:
vertically changing a focal point at a plurality of depths of the solution to determine whether the artificial particles are present at the depths of the solution, wherein the depths are selected based on a settling speed and the settling time of the artificial particles;
in response to the determination of the presence of the artificial particles at one or more presence depths, determining a cellular sample distribution at the one or more presence depths;
determining whether the cellular sample distribution is below a threshold density; and
in response to the determination that the cellular sample distribution is below the threshold density, providing an error message regarding the cellular sample distribution at a user interface.

15. The method of claim 1, wherein the solution is provided on a sample holder operably disposed of in a well or a chamber of the microscopy device, wherein the sample holder is a cartridge, a microslide, or a lab-on-chip.

16. A system for use with a biological sample, the system comprising:
a microscopy device;
a diluent;
a sample holder; and
one or more of lyophilized cakes comprising artificial particles,
wherein:
the lyophilized cakes and the biological sample are mixed with the diluent in the sample holder to form a solution;
the biological sample is imaged with the artificial particles as reference markers using the microscopy device; and
a settling time of the artificial particles is shorter than or equal to a settling time of the biological sample.

17. The system of claim 16, wherein the imaging comprises:
arranging a focal point of the microscopy device in one or more depths of interest;
capturing an in-focus image of the artificial particles at the one or more depths of interest;
in response to capturing the in-focus image of the artificial particles, determining whether the biological sample is present at the one or more depths of interest;
in response to the determination of the biological sample at the one or more depths of interest, capturing an in-focus image of the biological sample to determine one or more imaging events, the imaging events comprising a settlement of the biological sample, a confirmation of reagent, a confirmation of acellular sample, a confirmation of magnification, estimation of incubation time; and
wherein the one or more depths of interest is around the bottom of the solution, around the top of the solution, or depths determined based on a settling speed and a settling time of the artificial particles.

18. The system of claim 16, wherein:
the artificial particles are one or more of polymeric beads or inorganic beads, wherein the polymeric beads are polystyrene beads, acrylic beads, polyethylene beads, polypropylene beads, polyacrylate beads, or Polymethylhydrosiloxane (PMHS), and the inorganic beads are glass beads, silicon dioxide beads, or metal beads; and
the system further comprises an imaging algorithm, wherein the imaging algorithm adopts one or more offsets to identify the artificial particles and the biological sample, and the offsets are based on one or more differences between the artificial particles and the biological sample in size, fluorescent wavelength, or surface morphology.

19. The system of claim 16, wherein the lyophilized cakes further comprise fluorescent dyes.

20. The system of claim 16, wherein:
a lyophilization of the lyophilized cakes comprising one or more freezing cycles and one or more drying cycles, wherein temperatures of the freezing cycles is greater than or equal to −50° C. and less than or equal to 5° C., and temperatures of the drying cycles is greater than or equal to −50° C. and less than or equal to 25° C.; and
the lyophilized cakes dispense in the solution in absence of aggregation.
